(19) United States
(12) Patent Application Publication
(10) Pub. No.: US 2008/0038353 A1
(43) Pub. Date: Feb. 14, 2008

(54) Polymer Based Nano-Carriers For The Solubilization And Delivery Of Hydrophobic Drugs

(75) Inventors: Afsaneh Lavasanifar, Edmonton (CA); Glen Kwon, Waunakee, WI (US)

Correspondence Address: BENNETT JONES;C/O MS ROSEANN CALDWELL, 4500 BANKERS HALL EAST, 855 - 2ND STREET, SW, CALGARY, AB, T2P 4K7, CA

(73) Assignees: THE GOVERNORS OF THE UNIVERSITY OF ALBERTA, Edmonton, AB (CA); WISCONSIN ALUMNI RESEARCH FOUNDATION, Madison, WI (US)

(21) Appl. No.: 11/569,719

(22) PCT Filed: Jun. 2, 2005
(86) PCT No.: PCT/CA05/00835

§ 371 Date: Mar. 16, 2007

Related U.S. Application Data
(60) Provisional application No. 60/576,697, filed on Jun. 2, 2004.

(51) Int. Cl. C08G 63/664; A61K 31/335; A61K 31/34; A61K 31/35; A61K 31/40; A61K 31/55; A61K 31/56; A61K 31/655; A61K 31/70; A61K 31/711; A61K 38/00; A61K 38/13; A61K 9/00; A61P 37/06; C08G 63/08

(52) U.S. Cl. 424/486; 514/150; 514/182; 514/2; 514/221; 514/23; 514/418; 514/44; 514/449; 514/452; 514/459; 514/469; 514/9; 525/165

(57) ABSTRACT

The present invention is in the field of polymer-based nano-carriers for the solubilization and delivery of hydrophobic drugs, and relates to methods of making said carriers, and to pharmaceutical compositions comprising said carriers. Novel PEO-b-PCL micelles and micelles containing cyclosporine A or analogs thereof are provided as well as a novel method for making said micelles that reduces aggregation and enhances delivery, the toxicity profile and biodistribution of hydrophobic drugs.

### FIELD OF THE INVENTION

The present invention is in the field of polymer-based nano-carriers for the solubilization and delivery of hydrophobic drugs and relates to methods of making said carriers, and to pharmaceutical compositions comprising said carriers.

### BACKGROUND OF THE INVENTION

Cyclosporine is a neutral, lipophilic cyclic endecapeptide with very low water solubility (23 μg/ml). cyclosporine is the leading immunosuppressive agent used primarily to reduce the incidence of graft rejection in recipients of transplanted organs. In effect, the introduction of cyclosporine has greatly improved the chances for long-term survival of the transplanted organ. Nearly 50,000 new patients worldwide who receive transplanted organs annually and more than 200,000 transplant recipients in North America and Europe depend on daily cyclosporine therapy to prevent organ rejection. Acute and chronic nephrotoxicity is the most common side effect of cyclosporine.

Cyclosporine is one of the most effective modulators of drug resistance in cancer, as well. Unlike other modulators of drug resistance, cyclosporine potentiates chemotherapeutic drug cytotoxicity in certain sensitive cell lines. Acute and chronic toxicities associated with the co-administration of cyclosporine formulations and anticancer agents have limited the clinical effectiveness of cyclosporine in cancer patients. Cyclosporine blocks the biliary and renal clearance of anticancer agents through inhibition of P-glycoprotein. It also inhibits cytochrome P450 which is involved in the metabolism of a several anticancer agents.

In addition cyclosporine has been used to treat psoriasis, Behcet's disease, inflammatory bowel disease, and rheumatoid arthritis.

The first approved formulation of cyclosporine, Sandimmune®, was introduced in Europe in 1981 and then approved by Food and Drug Administration in the U.S. in 1983. Cyclosporine is produced by Sandoz/Novartis and different generic pharmaceutical companies in two forms:
1. Cyclosporine for injection (Sandimmune® iv by Novartis) which uses ethyl alcohol and cremophor EL as major components for solubilization of cyclosporine. Cremophor EL and alcohol both have limitations in terms of safety of the parental dosage form. Part of cyclosporine nephrotoxicity is attributed to Cremophor EL. Hypersensitivity reactions to cyclosporine after intravenous administration of Sandimmune® are also due to Cremophor EL.
2. Cyclosporine oral solution or soft gelatin capsules (Neoral® by Novartis) which is a micro emulsion formulation of cyclosporine. The micro emulsion is miscible in water and provides improved pharmacokinetic bioavailability for oral administration of cyclosporine. A lower incidence of adverse events is observed for patients who received Neoral® compared to those maintained with Sandimmune®. The dose of the microemulsion is 10 to 20 percent lower than standard cyclosporine. Limited stability and shelf life of the micro emulsion formulation is of concern. Both formulations provide instant release forms of cyclosporine after administration.

Because cyclosporine and other hydrophobic drugs, or water insoluble or poorly soluble modulators of drug resistance such as PSC 833 (a cyclosporine A analog) are often hydrophobic and toxic to humans, and because they may enhance the toxicity of anticancer agents in co-administration, there is a need for the development of pharmaceutical compositions comprising formulations which are improved in relative toxicity to the patient and in release properties for such agents.

One method of drug delivery that has been explored to address these problems uses amphiphilic block copolymers that self-assemble in aqueous environments to form polymeric micelles. These micelles can be loaded with hydrophobic drugs and used to enhance delivery of hydrophobic drugs. Block copolymer micelles can increase the therapeutic efficacy of drugs by preventing rapid drug clearance, decreasing their systemic toxicity and enhancing release properties as well as improving their biodistribution and avoiding immune detection and preventing immune reaction.

Various polymer components have been used to deliver biologics and drugs to cells. Zastre and colleagues have used low molecular weight methoxy poly(ethylene glycol)-block-polycaprolactone (PEO-b-PCL)diblock copolymers to deliver a P-glycoprotein substrate to caco-2 cells¹ and Kim et al. have used taxol-loaded block copolymer nanospheres composed of methoxy poly(ethylene glycol) and poly(epsilon-caprolactone) as novel anticancer drug carriers².

U.S. Pat. No. 6,469,132, entitled “Diblock copolymer and use thereof in a micellar drug delivery system” describes a PEO-b-PCL micellar system for the delivery of biologically active agents. Although claiming block copolymers of PEO-b-PCL with higher molecular weight, the patent discloses only a method for making micelles with significantly lower PEO and PCL molecular weights [PEO MW<4000 Daltons, PCL MW<3000 Daltons] U.S. Pat. No. 6,469,132 also uses the organic solvent dimethyl formamide (DMF) for loading the micelle preparation. DMF is highly toxic, causing liver damage and embryo-toxicity. It is also a suspected carcinogen. Insufficient removal of DMF can hence pose serious toxicity problems. The micelles of U.S. Pat. No. 6,469,132 have a problem with micelle aggregation and the use of a potentially toxic solvent.

U.S. Pat. No. 6,322,805, entitled “Biodegradable polymeric micelle-type drug composition and method for the preparation thereof’ describes methods for the encapsulation of cyclosporine in nanoparticles of PEO-b-PCL. The final size of the loaded nanoparticles (micelles) is not provided in patent U.S. Pat. No. 6,322,805. The molecular weight of the amphiphilic block copolymer used to form the micelles has a molecular weight in the range of about 1430 to 6000 Daltons. The low molecular weight of the block copolymers results in water solubility of the polymer. However the low molecular weight of the PEO block may also lead to aggregation in vitro, compromising the stability of the particles in vivo.

One of the challenges in optimizing micellar drug delivery is particle size, while having sufficient amount of drug loaded in the micelle for drug delivery. Smaller particles can escape uptake by reticuloendothelial system (phagocytes of the immune system) more efficiently leading to reduced clearance and increased blood concentrations of the encapsulated drug in vivo.

There is a need for improved carriers of hydrophobic drugs, for improved micelle carriers that minimize aggregation have a better toxicity profile, good drug load and superior biodistribution.

### SUMMARY OF THE INVENTION

The present invention circumvents many of the problems present in the prior art and provides methods and materials for the delivery of water insoluble or hydrophobic drugs. The invention results in increased control of drug release and less toxicity due to the use of less toxic drug formulations and superior biodistribution as a result of reduced micelle aggregation. An enhanced micelle drug delivery system for hydrophobic drugs and a new formulation for cyclosporine and cyclosporine analogs such as PSC 833 that may be used for immunosuppression or modulation of drug resistance has been developed. Further, a new formulation for amiodarone, a benzofuran derivative that blocks both the α- and β-adrenoreceptors, has also been developed.

Accordingly, the present invention includes a new formulation or drug delivery vehicle for the administration of hydrophobic biologically active agents in micelles formed from self-assembly of poly(ethylene oxide)-b-poly(ε-caprolactone) (PEO-b-PCL). In one embodiment the micelles are composed of copolymers of high molecular weight. In one embodiment, the PEO-b-PCL copolymer used in micelle formation of the invention exhibits a molecular weight of greater than 6000 Daltons and in another embodiment exhibits a molecular weight of greater than 10000 Daltons. In one embodiment the micelles of the invention are formed using copolymers of molecular weight of about 7000-29000 Daltons. In another embodiment, formation of the micelles involves the use of a water miscible solvent. In one embodiment, the resultant micelles have an average diameter of less than 100 nm in the absence of agent, suitably 55-90 nm in size. In another embodiment, agent loaded micelles have an average diameter of less than 200 nm, suitably 60-125 nm in size. In one embodiment, more than one type of biologically active agent is loaded into the micelle. In another embodiment the micelle of said invention includes an inhibitor of P-glycoprotein or a modulator of drug resistance.

The invention also includes a pharmaceutical composition and method of treatment that can be used, for example, to reduce graft rejection of transplanted organs or tissues. In another aspect, the composition of said invention is used, for example, to treat cancer or resistant forms of cancer or infectious diseases. In another aspect, the composition of the present invention is used, for example, to treat diseases that benefit from a blocking of the α- and β-adrenoreceptors, for example to treat angina and arrhythmias. Further, the compositions of the invention may be administered, for example, by intravenous injection, or by oral or pulmonary routes. In addition, the micelles and compositions of the invention are used to enhance the permeability of drugs across the blood brain barrier or in the gastrointestinal tract.

The present invention also includes a method for preparing PEO-b-PCL micelles comprising assembling a PEO-b-PCL block co-polymer of the present invention in a suitable aqueous medium under conditions sufficient to minimize aggregation.

In an embodiment of the present invention, the method for preparing PEO-b-PCL micelles comprises
a. obtaining a solution of PEO-b-PCL block copolymers in a water miscible solvent;
b. combining the solution of PEO-b-PCL block copolymers with a suitable aqueous medium under conditions sufficient to minimize aggregation; and
c. removing the water miscible organic solvent.

Other features and advantages of the present invention will become apparent from the following detailed description. For instance, although not wishing to be bound to any particular theory, the use of higher molecular weight PEO polymers in the structure of block copolymer results in less aggregation of micelle particles and modified biodistribution. In another aspect, the invention provides a micelle of a small size with an enhanced drug load which is better able to escape immune detection and avoid clearance leading to enhanced blood concentration. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1A is a schematic representation of the synthetic formation of PEO-b-PCL. FIG. 1B is a spectral trace of a typical ¹H NMR spectrum of PEO-b-PCL.

FIG. 2A is a graph showing the effect of temperature on the percentage of ε-caprolactone conversion to PCL in the synthesis of PEO-b-PCL diblock copolymers at a catalyst to monomer molar ratio of 0.002. FIG. 2B is a graph showing the effect of catalyst concentration on the percentage of ε-caprolactone conversion to PCL in the synthesis of PEO-b-PCL diblock copolymer at 140° C. after 4 hours of reaction.

FIG. 3 shows the fluorescence emission spectrum of 1,3-(1,1′-dipyrenyl)propane in micellar solutions of PEO-b-PCL (different PCL chain lengths) in comparison to Cremophor EL as an indication of micellar core viscosity.

FIG. 4 is a graph showing the effect of solubilizing vehicle on the in vitro rate of CsA release. Data is presented as mean±SD (n=3).

FIG. 5 is a graph showing CsA concentration-time profile in whole blood after intravenous injection of CremophorEL formulation (Sandimmune®) and CsA-loaded polymeric micelles. Micellar data is normalized to injected dose of Cremophor EL and presented as mean±SD (n=4).

FIG. 6 shows CsA concentration-time profiles in A) blood, B) plasma, C) kidney, D) liver, E) spleen and F) heart following intravenous administration of CsA in polymeric micellar formulation in comparison to CsA in Cremophor EL (Sandimmune®) formulation. Micellar data is normalized to injected dose of Cremophor EL and presented as mean±SD (n=4). * Denotes a significant difference between concentrations at that time point (p<0.05, unpaired t test) FIG. 7 shows tissue to plasma concentration ratio (Kₚ) time profiles of CsA after intravenous administration of its polymeric micellar formulation and Sandimmune® (CsA formulation in Cremophor EL). The data presents A) kidney; B) liver; C) spleen, and D) heart to plasma ratios. Micellar data is normalized to injected dose of Cremophor EL and presented as mean±SD (n=4).

### DETAILED DESCRIPTION OF THE INVENTION

The present invention describes a system of drug delivery for hydrophobic or toxic biologically active agents such as amiodarone and cyclosporine or analogs thereof, using PEO-b-PCL micelles and describes new formulations of PEO-b-PCL micelles loaded with one or more biologically active agents.

Definitions

Amiodarone—a benzofuran derivative that blocks both the α- and β-adrenoreceptors.

Block copolymer—A polymer whose molecules consist of blocks of different species that are connected linearly.

Critical Micelle Concentration (CMC)—The concentration above which amphiphilic molecules including block copolymers self assemble and form a supramolecular core/shell structure i.e., micelles.

CsA—cyclosporine A

Cyclosporine (also cyclosporin)—mycotoxin that suppresses the immune system and includes cyclosporine A, cyclosporine C, cyclosporine D, cyclosporine G as well as analogs, such as analogs, derivatives and pharmaceutical acceptable salts thereof.

Cyclosporine analogs—compounds that have a similar structure and function to cyclosporine such as PSC 833.

Biologically active agent/Drug—used interchangeably herein and includes, for example, any organic or inorganic small molecule compound, polymeric species (including nucleic acids (DNA or RNA), proteins, peptides, carbohydrates and derivatives thereof), lipids and mixtures thereof, wherein said drug or agent is administered in vivo (in humans or animals) for the treatment of any disease, condition or disorder.

DMF—dimethyl formamide.

Effective amount or sufficient amount of an agent—that amount sufficient to effect beneficial or desired results, including clinical results, and, as such, an “effective amount” depends upon the context in which it is being applied. For example, in the context of administering an agent that is effective for treating disease, an effective amount of an agent is, for example, an amount sufficient to achieve such treatment as compared to the response obtained without administration of the agent.

Hydrophobic drug or biologically active agent—a drug or biologically active agent that has a lack of affinity for water, does not absorb water and precipitates at concentrations greater than 10 mg/ml.

Micellization—A colloidal aggregation of amphipathic molecules, which occurs at a defined concentration known as the critical micelle concentration (CMC, see above).

Molecular Weight—as used herein refers to average molecular weight and the units can be in Daltons or g.mol⁻¹.

MRT—mean residence time.

Palliating a disease or disorder means that the extent and/or undesirable clinical manifestations of a disorder or a disease state are lessened and/or time course of the progression is slowed or lengthened, as compared to not treating the disorder.

PCL—poly (ε-caprolactone).

PEO—poly (ethylene oxide) also known as poly(ethylene glycol) or PEG.

PEO-b-PCL—poly (ethylene oxide)—block—poly (ε-caprolactone).

PSC 833—cyclosporine analog (Novartis Pharmaceuticals).

Pharmaceutically acceptable—suitable for or compatible with the treatment of subjects, in particular humans.

Subjects—includes all members of the animal kingdom and most suitably is human.

Treatment or treating—an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. Treatment or treating can also mean prolonging survival as compared to expected survival if not receiving treatment.

Water-insoluble—molecules or materials which are incapable or poorly capable of dissolving in water; a drug that precipitates at concentrations greater than 10 mg/ml in water.

Water miscible organic solvent—organic solvents that can be mixed with water and form one phase (not separated) such as acetonitrile, ethylacetate, methanol, ethanol, propylene glycol, tetrahydrofuran (THF), etc.

Description

Amphiphilic block copolymers such as PEO-b-PCL self assemble to core/shell structures, namely polymeric micelles, in aqueous environments and effectively encapsulate hydrophobic biologically active agents. PEO-b-PCL micelles are unique among drug carriers owing to the biocompatibility and biodegradability of the PEO and PCL block, nanoscopic dimensions and distinct properties of the PEO/PCL core/shell structure.

The small size of polymeric micelles and the PEO shell can help the carrier to stay unrecognized, as self, in the biological system. Other advantages associated with nanoscopic dimensions of polymeric micelles include the ease of sterilization via filtration and safety of administration. The semicrystalline and hydrophobic PCL core of the PEO-b-PCL micelles can take up, protect and retain hydrophobic biologically active agents such as cyclosporine, leading to improved solubility/stability of the agents in vivo, controlled release, reduced toxicity and attenuated pharmacokinetic interaction with other substrates of, for example, p-glycoprotein including anticancer agents.

The present invention also includes compositions comprising PEO-b-PCL micelles and biologically active agents, a method of making said compositions and a method of using said compositions in the treatment of disease.

In one embodiment the present invention includes a safe replacement for toxic ingredients in the commercial formulations of cyclosporine, e.g. Cremophor EL and ethanol, while providing cyclosporine levels in aqueous media that are clinically relevant, a control over the rate of drug release, a change in the normal biodistribution of cyclosporine, taking it away from its site of toxicity, i.e., kidneys, and providing improved delivery of the drug to its site of activity. In another embodiment of the present invention there is included a formulation comprising amiodarone encapsulated within a PEO-b-PCL micelle.

PEO-b-PCL Copolymer

The present invention describes a method of making a PEO-b-PCL block copolymer. The methoxy poly(ethylene oxide) (methoxyPEO) used in the formation of PEO-b-PCL block copolymer, have a high average molecular weight, between about 5000-20000 Daltons, such as about 5000 Daltons. Sources of methoxyPEO known in the art include those obtainable from Sigma Chemical Company USA. (Catalog number M-7268).

ε-Caprolactone monomers used in the present invention have an average molecular weight of 114 Daltons and sources of ε-caprolactone known in the art include those obtainable from Aldrich Chemical Company Inc. USA. Once polymerized with PEO, as described below, the molecular weight of the ε-caprolactone portion of the co-polymer is greater than about 5000 Daltons, suitably about 5000-24000 Daltons. In another embodiment the molecular weight of the ε-caprolactone portion is about 13000 Daltons.

The PEO-b-PCL block copolymer may be prepared using methods known in the art^{3,4,5,6}. In general, PEO, for example methoxy-PEO, and ε-caprolactone are combined in the presence of a catalyst, for example a tin or aluminum alkoxide, such as stannous octoate or aluminum tri-isopropoxide, suitably stannous octoate, and the reaction mixture heated, for example at a temperature of about 120° C. to about 180° C., suitably about 130° C. to about 170° C., more suitably about 140° C. to about 160° C., for about 2 to about 8 hours, suitably about 3 to about 7 hours, more suitably about 4 to about 6 hours. Typically, higher reaction temperatures, result in shorter reaction times to achieve sufficient conversion of ε-caprolactone. In an embodiment of the invention, a reaction time of about 3 hours and a temperature of about 160° C. is used. In the preparation method used herein, the PEO was used as an initiator, but higher and lower molecular weights of methoxy PEO or amino methoxy PEO can also be used.

In one aspect of the invention the molar ratio of ε-caprolactone monomer to initiator is greater than about 8:1. In another embodiment the ratio of ε-caprolactone monomer to initiator is between about 50:1 to 250:1. In another embodiment the ratio of ε-caprolactone monomer to initiator is 44:1, 114:1 or 210:1.

In one aspect of the invention, the PEO-b-PCL molecular weight is greater than about 6000 Daltons. In another embodiment, the molecular weight of the PEO-b-PCL is between 7000-29000 Daltons. In another embodiment the PEO-b-PCL molecular weight is between about 10,000-29,000. In another embodiment the PEO-b-PCL molecular weight is about 18000 Daltons.

Micelle Formation

The present invention includes a method for preparing PEO-b-PCL micelles comprising assembling a PEO-b-PCL block co-polymer of the present invention in a suitable aqueous medium under conditions sufficient to minimize aggregation.

In an embodiment of the present invention, the method for preparing PEO-b-PCL micelles comprises
a. obtaining a solution of PEO-b-PCL block copolymers in a water miscible solvent;
b. combining the solution of PEO-b-PCL block copolymers with a suitable aqueous medium under conditions sufficient to minimize aggregation; and
c. removing the water miscible organic solvent.

The PEO-b-PCL block copolymers are suitably those according to the present invention as described hereinabove.

The water miscible solvent is suitably any water miscible organic solvent with low or no known toxicity including, but not limited to, ethanol, acetone, ethyl acetate and acetonitrile. In embodiment of the invention, the water miscible solvent is acetone or acetonitrile, suitably acetone.

The aqueous medium may be any suitable polar solvent, for example water, suitably, distilled water, or any aqueous medium suitable for in vivo administration to subjects, in particular human subjects, for example normal saline, 5% dextrose or isotonic sucrose.

The conditions that influence aggregation of the micelles include, for example, the molecular weight of the block copolymer, the ratio of the solvent phase to the aqueous phase, the identity of the aqueous medium, the identity of the water miscible solvent and the method of combining the two phases. A person skilled in the art would be able to adjust these conditions to minimize the formation of aggregates. Formation of aggregates can be monitored using, for example, light scattering techniques as described in the Examples below.

The ratio of the solvent phase to the aqueous phase can be adjusted to adjust the size of the resulting micelles. For example, using a lower proportion of the solvent phase results in smaller micelles. Suitably the ratio of solvent phase to aqueous phase is in the range of about 1:1 to about 1:10, more suitably about 1:2 to about 1:6.

The aqueous medium and the solution of the PEO-b-PCL block copolymers may be combined in any order. In an embodiment of the invention, the solution of the PEO-b-PCL block copolymers is added to the aqueous medium in small aliquots or small volumes as compared to the amount of aqueous medium, such as by drop-wise addition.

The water miscible solvent may be removed using any known technique, such as, by dialysis or solvent evaporation. In an embodiment of the invention the solvent is removed by evaporation under reduced pressure, suitably at about 20° C. to about 30° C.

In one embodiment, the invention includes an unloaded or empty micelle produced by the method of the present invention. In one embodiment the resultant micelles have an average diameter of less than 100 nm. In another embodiment, the unloaded micelles have an average diameter of between about 55 nm and about 90 nm.

If biologically active agents are to be loaded into the micelles, said agents, suitably hydrophobic drugs, are added to the block copolymer water miscible solution at step (a) or before step (b) above, and the said solution is added to water or other polar solvent in a manner that reduces aggregate formation, such as a drop-wise addition of the block copolymer dissolved in a water miscible solvent to water or other polar solvent. In one embodiment, the agent-loaded into the micelle also comprises as suitable pharmaceutical carrier. Once again micellar characteristics and agent-loading levels may be optimized through changes in the self assembly process. For example, for CsA loading in PEO-b-PCL micelles, optimum drug solublization and micellar size can be achieved with the addition of an acetone solution comprising CsA and PEO-b-PCL to water at a final organic to aqueous phase ratio of about 1:6.

In one embodiment, one biologically active agent, optionally with one or more pharmaceutically acceptable carriers, is loaded in the micelle, in another embodiment, more than one type of biologically active agent, optionally with one or more pharmaceutically acceptable carriers, are loaded into the micelle. This is especially useful in combination therapies such as cancer. In one embodiment, the invention provides drug-loaded micelles having an average diameter that is less than 200 nm. In another embodiment the drug loaded micelles have an average diameter between about 60 nm and about 125 nm. In a further embodiment the drug loaded micelles have an average diameter of about 100 nm. In another embodiment, the drug-loaded micelles have an average diameter of about 120 nm.

The biologically active agent may be any such agent that one wishes to load into a micelle, in particular for administration to subjects. In an embodiment of the invention the biologically active agent is any organic or inorganic small molecule compound, polymeric species (including nucleic acids (DNA or RNA), proteins, peptides, carbohydrates and derivatives thereof), lipids and mixtures thereof, wherein said drug or agent is administered in vivo (in humans or animals) for the treatment of any disease, condition or disorder. In a further embodiment of the invention, the biologically active agent is a hydrophobic drug, including but not limited to cyclosporin A, PSC 833, amiodarone, amphotercin B, nystatine, diazepam, verapamil, indomethacin, taxol, rapamycin, etoposide and estradiol. In still further embodiments of the invention, the biologically active agent is cyclosporine A and/or one of its analogs, for example PSC 833 or amiodarone.

In one embodiment the invention includes a PEO-b-PCL micelle that is unloaded or loaded with a biologically active agent and is water insoluble. In another embodiment, the micelles have an average molecular weight of greater than 6000 Daltons. In another embodiment, the invention includes PEO-b-PCL micelles that have a PEO and PCL block length of about 5000 Daltons for PEO and 2000-24000 Daltons or 13000 Daltons for PCL. Not wishing to be bound by a theory, higher PEO block lengths may avoid aggregation of block copolymer micelles in vitro and lead to better stability in vivo.

Pharmaceutical Compositions

The polymeric micelles of the present invention were shown to modify the pharmacokinetics and tissue distribution of cyclosporine A (CsA).⁷ Drug-loaded methoxy poly(ethylene oxide)-b-poly(ε-caprolactone) (PEO-b-PCL) micellar solutions in isotonic medium were prepared and administered intravenously to healthy Sprague-Dawley rats. Blood and tissues were harvested and assayed for CsA, and resultant pharmacokinetic parameters and tissue distribution of CsA in its polymeric micellar formulation were compared to its commercially available intravenous formulation (Sandimmune®). In the pharmacokinetic assessment, a 6.1 fold increase in the area under the blood concentration versus time curve (AUC) was observed for CsA when given as polymeric micellar formulation as compared to Sandimmune®. The volume of distribution and clearance of CsA as PEO-b-PCL formulation were observed to be 10.0 and 7.6 fold lower, respectively, compared to the commercial formulation. No significant differences in t_{1/2} or MRT could be detected. In the biodistribution study, analysis of tissue samples indicated that the mean AUC of CsA in polymeric micelles was lower in liver, spleen and kidney (1.5, 2.1 and 1.4-fold, respectively). Similar to the pharmacokinetic study in these rats the polymeric micellar formulation gave rise to 5.7 and 4.9-fold increases in the AUC of CsA in blood and plasma, respectively. These results show that PEO-b-PCL micelles can effectively solubilize CsA, at the same time confining CsA to the blood circulation and restricting its access to tissues such as kidney, perhaps limiting the onset of toxicity.

The micelles of the invention may, therefore, be suitably formulated into pharmaceutical compositions for administration to human subjects in a biologically compatible form suitable for administration in vivo. Accordingly, in another aspect, the present invention includes a pharmaceutical composition comprising micelles of the invention, in admixture with a suitable diluent or carrier.

The compositions containing the micelles of the invention can be prepared by known methods for the preparation of pharmaceutically acceptable compositions which can be administered to subjects, such that an effective quantity of the biologically active agent within the micelles is combined in a mixture with a pharmaceutically acceptable vehicle. Suitable vehicles are described, for example, in Remington's Pharmaceutical Sciences (2003-20th edition), in The United States Pharmacopeia: The National Formulary (USP 24 NF19) published in 1999 and in the Handbook of Pharmaceutical Additives (compiled by Michael and Irene Ash, Gower Publishing Limited, Aldershot, England (1995)). On this basis, the compositions include, albeit not exclusively, solutions of the micelles in association with one or more pharmaceutically acceptable vehicles or diluents, and contained in buffered solutions with a suitable pH and iso-osmotic with the physiological fluids. In this regard, reference can be made to U.S. Pat. No. 5,843,456. As will also be appreciated by those skilled, administration of substances described herein may be by an inactive viral carrier. In one aspect the pharmaceutical compositions of the invention can be used to enhance biodistribution and drug delivery of hydrophobic drugs.

In accordance with the methods of the invention, the described micelles of the invention, may be administered to a subject in a variety of forms depending on the selected route of administration, as will be understood by those skilled in the art. The micelles of the invention may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A micelle of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the micelle of the invention may be incorporated with excipient and used in the form of ingestible tablets, buccal tablets, troches, capsules, elixirs, suspensions, syrups, wafers, and the like.

A micelle of the invention may also be administered parenterally. Solutions of a micelle of the invention can be prepared in water suitably mixed with suitable excipients. Under ordinary conditions of storage and use, these preparations contain a preservative to prevent the growth of microorganisms. A person skilled in the art would know how to prepare suitable formulations.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The compounds of the invention, may be administered to an animal alone or in combination with pharmaceutically acceptable carriers, as noted above, the proportion of which is determined by the solubility and chemical nature of the compound, chosen route of administration and standard pharmaceutical practice.

In an embodiment of the invention, the pharmaceutical compositions are administered in a convenient manner such as by direct application to the infected site, e.g. by injection (subcutaneous, intravenous, etc.). In case of respiratory infections, it may be desirable to administer the micelles of the invention and compositions comprising same, through known techniques in the art, for example by inhalation. Depending on the route of administration (e.g. injection, oral or inhalation, etc . . . ), the pharmaceutical compositions or micelles or biologically active agents in the micelles of the invention may be coated in a material to protect the micelles or agents from the action of enzymes, acids and other natural conditions that may inactivate the compound.

In addition to pharmaceutical compositions, compositions for non-pharmaceutical purposes are also included within the scope of the present invention, such as for diagnostic or research tools. In one embodiment, the biologically active agents or micelles comprising said drugs can be labeled with labels known in the art, such as florescent or radio-labels or the like.

Applications

The present invention includes a delivery system that can be used to deliver biologically active agents or formulations or pharmaceutical compositions. In one embodiment, the invention includes the delivery of hydrophobic biologically active agents. In another embodiment, the invention includes delivery of hydrophobic biologically active agents by loading them into micelles comprising a hydrophobic core and a hydrophilic outer surface, thus improving their delivery in aqueous mediums, such as blood and body fluids. In other aspect, the invention includes the delivery of biologically active agents that can reduce their toxicity profile. The invention also includes a method for reducing aggregation of the micelle delivery vesicles of the invention. As such, it provides for better biodistribution of biologically active agents resulting in decreased toxicity and/or improved therapeutic efficacy.

Another aspect of the invention includes a method of delivering biologically active agents to treat a disease, condition or disorder in a subject in need thereof comprising administering an effect amount of an agent-loaded micelle of the invention to said subject. In one embodiment, the agent is cyclosporine, CsA or analog thereof. Accordingly, the disease, condition or disorder is one that benefits from the administration of CsA or an analog thereof. Such diseases, conditions or disorders are known in the art and include use as an immunosuppressant, for instance in mammals receiving an organ or tissue transplant. In another aspect, the disease, condition or disorder is cancer or drug resistant cancers, infectious disease or an autoimmune disease. In a further embodiment of the invention, the agent amiodarone and the disease, condition or disorder is one that benefits from the administration of amiodarone. Such diseases, conditions or disorders are known in the art and include angina and arrhythmias.

The dosage of the micelles of the invention can vary depending on many factors such as the pharmacodynamic properties of the micelle, the biologically active agent, the rate of release of the agent from the micelles, the mode of administration, the age, health and weight of the recipient, the nature and extent of the symptoms, the frequency of the treatment and the type of concurrent treatment, if any, and the clearance rate of the agent and/or micelle in the subject to be treated. One of skill in the art can determine the appropriate dosage based on the above factors. The micelles may be administered initially in a suitable dosage that may be adjusted as required, depending on the clinical response. For ex vivo treatment of cells over a short period, for example for 30 minutes to 1 hour or longer, higher doses of micelles may be used than for long term in vivo therapy.

The micelles of the invention, can be used alone or in combination with other agents that treat the same and/or another condition, disease or disorder.

In another embodiment, where either or both the micelle or biologically active agent is labeled, one can conduct in vivo or in vitro studies for determining optimal dose ranges, drug loading concentrations and size of micelles and targeted drug delivery for a variety of diseases.

The following non-limiting examples are illustrative of the present invention:

### EXAMPLES

Materials and Methods

Stannous octoate (96% pure) and biphenyl (99.5%) were obtained from Aldrich (Milwaukee, Wis., USA). Methoxy polyethylene oxide (average molecular weight of 5000 gmole⁻¹), ε-caprolactone, amiodarone HCl (98%), and cyclosporine A (CsA) were purchased from Sigma (St. Louis, Mo., USA). Acetonitrile and chloroform were supplied by Fisher Scientific (Nepean, Ontario, Canada). All other chemicals were reagent grade. Sodium chloride injection 0.9% was obtained from Abbott Laboratories (Montreal, PQ, Canada). Heparin sodium injection, 10,000 IU/mL was purchased from Leo Pharma Inc. (Thornhill, ON, Canada).

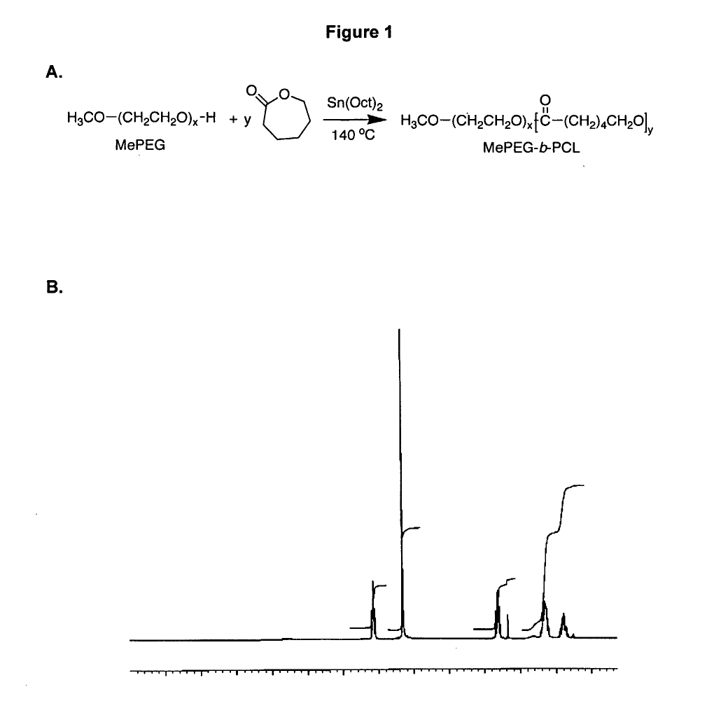

Synthesis and characterization of polyethylene oxide-block-poly(ε-caprolactone) (PEO-b-PCL) block copolymers—PEO-b-PCL block copolymers were synthesized by ring opening polymerization of ε-caprolactone using methoxy polyethylene oxide (Molecular Weight of 5000 g.mol⁻¹) as an initiator and stannous octoate as a catalyst (FIG. 1A). Methoxy PEO (5 g), ε-caprolactone and stannous octoate were added to a previously flamed 10 ml ampoule, nitrogen purged, then sealed under vacuum. ¹H NMR spectrum of PEO-b-PCL in CDCl₃ at 300 MHz (FIG. 1B) was used to assess the conversion of ε-caprolactone monomer to PCL and determine the number average molecular weight of the block copolymers. The percentage of ε-caprolactone conversion to PCL was determined comparing peak intensity of —O—CH₂— (δ=4.223 ppm) for ε-caprolactone monomer to the intensity of the same peak for PCL (δ=4.075 ppm) in the ¹H NMR spectrum of PEO-b-PCL. The effect of reaction time, temperature, and catalyst concentration on the percentage of ε-caprolactone conversion to PCL was assessed to optimize the conditions of the reaction further. Different ε-caprolactone to methoxy PEO feed ratios were used to prepare PEO-b-PCL block copolymers with varying degrees of ε-caprolactone polymerization. The reaction product was dissolved in chloroform, precipitated, and washed with an excess of cold methanol, followed by centrifuge collection. Comparison of peak intensity of PEO (—CH₂CH₂O—, δ=3.65 ppm) to that of purified polymer PCL (—O—CH₂—, δ=4.075 ppm) provided an estimate for degree of ε-caprolactone polymerization. Prepared polymers were characterized for their average molecular weight and polydispersity by gel permeation chromatography. Samples (20 ml from 10 mg/ml polymer stock solutions in THF) were injected into a 4.6×300 mm Waters Styragel® HT4 column (Waters Inc., Milford, Mass.). A 1 mL/min THF mobile phase, set at 35° C., allowed the detection of the polymer using refractive index/light scattering detectors (Model 410, Walters Inc.). The calibration curve was prepared using polystyrene standards.

Preparation and characterization of PEO-b-PCL and Cremophor EL micelles—Assembly of block copolymers was achieved by co-solvent evaporation where PEO-b-PCL (10 mg) dissolved in acetone (0.5 mL) was added in a drop wise manner (1 drop/15 sec) to stirring distilled water (1 mL). The remaining acetone was removed by evaporation at room temperature under vacuum. Cremophor EL micelles were prepared by direct addition of the low molecular weight surfactant to water.

Mean diameter and polydispersity of self-assembled structures in aqueous media were defined by light scattering (3000HS_{A} Zetasizer Malvern, Zeta-Plus™ zeta potential analyzer, Malven Instrument Ltd., UK). The concentration of block copolymers and Cremophor EL was 10 mg/mL.

A change in the fluorescence excitation spectra of pyrene in the presence of varied concentrations of block copolymers was used to measure the critical micellar concentration (CMC). Pyrene was dissolved in acetone and added to 5 mL volumetric flasks to provide a concentration of 6×10⁻⁷ M in the final solutions. Acetone was then evaporated and replaced with aqueous polymeric micellar solutions with concentrations ranging from 0.05 to 500 mg/mL. Samples were heated at 65° C. for an hour, cooled to room temperature overnight, and deoxygenated with nitrogen gas prior to fluorescence measurements. The excitation spectrum of pyrene for each sample was obtained at room temperature using a Fluoromax DM-3000 spectrometer. The emission wavelength and excitation bandwidth were set at 390 and 4.25 nm, respectively. The intensity ratio of peaks at 337 nm to those at 333 nm was plotted against the logarithm of copolymer concentration to measure CMC⁸. This experiment was repeated for Cremophor EL solution at concentrations ranging from 0.5 mg/mL to 10 mg/mL.

The viscosity of the micellar cores was estimated by measuring excimer to monomer intensity ratio (Iₑ/Iₘ) from the emission spectra of 1,3-(1,1′-dipyrenyl)propane at 373 and 480 nm, respectively. 1,3-(1,1′-dipyrenyl)propane was dissolved in a known volume of chloroform to give a final concentration of 2×10⁻⁷ M. Chloroform was then evaporated and replaced with 5 mL of PEO-b-PCL or Cremophor EL micellar solutions at a concentration of 500 mg/mL and 10 mg/mL, respectively. Samples were heated at 65° C. for an hour and cooled to room temperature overnight. A stream of nitrogen gas was used to deoxygenate samples prior to fluorescence measurements. Emission spectrum of 1,3-(1,1′-dipyrenyl)propane was obtained at room temperature using an excitation wavelength of 333 nm, and an emission bandwidth was set at 4.25 nm⁷.

Optimization of the self-assembly and encapsulation process—As mentioned above, a co-solvent evaporation method was used for self assembly of PEO-b-PCL block copolymer and also for drug encapsulation. The applied organic solvent, the ratio of organic to the aqueous phase, and the order of addition of the phases in the co-solvent evaporation method were changed to optimize the preparation method in terms of micellar size and encapsulation efficiency. At the end of encapsulation process, the micellar solution was centrifuged at 11600×g for 5 minutes, to remove CsA precipitates. Mean diameter and poly dispersity of prepared polymeric micelles were defined by light scattering (3000HS_{A} Zetasizer Malvern, Zeta-Plus™ zeta potential analyzer, Malven Instrument Ltd., UK) at polymer concentration of 10 mg/mL in an aqueous media. An aliquot of the micellar solution in water was diluted with 3 times of acetonitrile to disrupt the micellar structure. Encapsulated levels of CsA were measured using reverse phase HPLC. The HPLC instrument consisted of a Chem Mate pump and auto-sampler. The HPLC system was equipped with an LC₁ column (Supleco) with a mobile phase of KH₂PO₄ (0.01 M), methanol and acetonitrile (25:50:25). The flow rate and column temperature were set at 1 mL/min and 65° C. (Eppendorf CH-30 column heater), respectively. CsA concentrations were determined by UV detection at 205 nm (Waters 481) after injection of 100 μL samples. The calibration samples were prepared at a concentration range of 0.1 to 10 μg/mL. Each experiment was conducted in triplicate. Cyclosporine loading and encapsulation efficiency were calculated from the following equations:

$$C sA loading ( w / w ) = \frac{amount of loaded CsA in mg}{amount of polymer in mg}$$

$$CsA loading ( M / M ) = \frac{moles of loaded CsA}{moles of polymer}$$

$$Encapsulat ion efficiency ( \% ) = \frac{amount of loaded CsA in mg}{amount of CsA added in mg} ⨯ 100$$

Cyclosporine A (CsA) encapsulation in PEO-b-PCL micelles by a co-solvent evaporation method—PEO-b-PCL block copolymer (20 mg) and CsA (2 or 3 mg) were dissolved in acetone (1 mL). This solution was added to 2 mL of either distilled water or normal saline in a drop wise manner. After 4 h of stirring at room temperature, vacuum was applied to ensure the complete removal of the organic solvent. The micellar solution was then centrifuged at 12,000 rpm for 5 min, to remove unloaded CsA. An identical method without CsA was used to prepare empty PEO-b-PCL micelles. The hydrodynamic diameter of PEO-b-PCL micelles with and without CsA was measured by dynamic light scattering (3000 HS_{A} Zetasizer Malvern, Zeta-Plus™ zeta potential analyzer, Malven Instrument Ltd., UK) at a polymer concentration of 10 mg/mL.

An aliquot of CsA loaded polymeric micelles was treated with 3 times volume of acetonitrile to disrupt the micellar structure. Level of encapsulated CsA was measured using a reverse phase HPLC method. Stock solutions of CsA (200 μg/mL) were prepared by dissolving 2 mg of CsA in 0.5 mL methanol, followed by addition of 9.5 mL distilled water, to create calibration standard curves. Biphenyl at a concentration of 0.5 mg/mL was used as internal standard. The HPLC instrument consisted of a Chem Mate pump and autosampler. 100 mL samples were injected to Hypersil C₁₈ column (250×4.60 mm; Phenomenex, Torrance, Calif., USA) with a mobile phase of acetonitrile: water (75:25, pH of 3.1 adjusted with phosphoric acid) running through column at a flow rate of 1.2 mL/min. The column was heated at 72° C. using an Eppendorf CH-30 column heater. CsA concentrations were estimated by UV detection at 210 nm (Waters, model 481). Cyclosporine loading and encapsulation efficiency were calculated using the formulae presented above.

In-vitro release study—CsA was dissolved in water at a concentration of 1 mg/mL with the aid of ethanol (40% v/v). Aqueous solutions of Cremophor EL and polymeric micellar formulations at a similar CsA concentration (corresponding to a concentration of 13 mg/mL for Cremophor EL and 8-11 mg/mL for block copolymers) were also prepared. One mL of each sample (ethanolic solution of CsA, Sandimmune®, or the polymeric micellar formulation) was placed in a Spectrapor dialysis bag (M.wt. cut-off=12000-14000 g.mol⁻¹). The dialysis bag was located in bovine serum albumin (BSA) solution (4% w/v, 60 mL) while stirring at 37° C. Samples of 0.5 mL were taken from the BSA solution after definite time intervals, and replaced by identical volume of fresh media. This experiment was also performed at room temperature. Level of released CsA in each sample was measured by HPLC after drug extraction. Amiodarone at a concentration of 10 mg/mL was used as internal standard. The internal standard solution (60 mL), deionized water (1.75 mL) and sodium hydroxide solution 1 M (200 mL) were added to release samples. Drug and internal standard were then extracted by ether-methanol 95:5 solution. After vortex mixing and centrifugation, the organic layer was removed and evaporated. The residue was solubilized in acetonitrile-0.5% v/v phosphoric acid (65:35) and washed with hexane. Samples of 30 μL from the aqueous lower layer were injected into the HPLC system, (Chem Mate pump and Auto sampler) which was equipped with an LC₁ column (Supleco) with a mobile phase of KH₂PO₄ (0.01 M), methanol and acetonitrile (25:50:25). The flow rate and column temperature were set at 1 mL/min and 65° C. (Eppendorf CH-30 column heater), respectively. CsA concentrations were determined by UV detection at 205 nm (Waters 481). The calibration samples were prepared at a concentration range of 0.1 to 10 mg/mL. Each experiment was conducted in triplicate. The percentage of released drug for organic solution of CsA, Sandimmune®, and polymeric micellar formulation was calculated and plotted versus time.

Assessing the pharmacokinetics of cyclosporine A formulations in healthy animal models—Sprague-Dawley male rats with body weights ranging from 250-300 g were used to examine the effect of solublizing vehicle on the pharmacokinetics of CsA. All rats were housed in a temperature-controlled room with a 12 h light/dark cycle for at least one week. The day before the pharmacokinetic experiment, the right jugular veins of all rats were catheterized with Micro-Renathane tubing (Braintree Scientific, Braintree, Mass.) under halothane anaesthesia, as previously described⁹. The cannula was flushed with 100 UmL⁻¹ heparin in 0.9% saline. After surgery, the rats were transferred to regular holding cages and allowed free access to water, but food was restricted overnight. The next morning, rats were transferred to metabolism cages. Animals were divided to two groups: control group received CsA in Cremophor EL formulation (Sandimmune®, Novartis) and test group received CsA in polymeric micellar formulation.

CsA in Cremophor EL formulation was diluted with normal saline for intravenous injection. The rats were given between 0.6 to 1 mL of each sample as intravenous bolus within 5 min. The actual dose of CsA injected to animals for Cremophor EL and polymeric micellar formulation was 5.0 and 2.5 mg/kg, respectively. Each sample was tested in 4 rats. After dosing, serial blood samples (˜0.2 mL) were obtained from the cannula for up to 24 h after. Sampling was performed at 5, 20, and 40 min, then 1, 2, 4, 6, 9, 12, and 24 h after drug administration. Between sampling, 0.2 mL of heparin 100 U/mL solution was used to maintain patency of the cannula. After collection, each blood sample was transferred to new glass tubes and stored at −20° C. until assessed for drug concentration by HPLC. Non-compartmental pharmacokinetic analysis was used to estimate pharmacokinetic parameters of area under the concentration-time curve (AUC), mean residence time (MRT), total clearance (CL), apparent volume of distribution at steady state (Vdₛₛ), and biological half-life (t_{1/2}) of CsA for each formulation.

Biodistribution Study—To assess the effect of formulation on the tissue distribution of CsA, rats were administered CsA in Cremophor EL formulation or CsA-loaded polymeric micelles at a similar dose to pharmacokinetic studies. Under light halothane anaesthesia, each rat received the drug as iv bolus doses by injection into the tail vein. At 5 min, or 1, 2, 6, 12, or 24 h after drug injection, each animal (n=4 for each time point) was anaesthetized and exsanguinated by cardiac puncture. Heart, spleen, liver, kidney, fat, brain as well as samples of whole blood and plasma, were collected. Tissue samples were blotted with paper towel, washed in ice cold saline, bottled to remove excess fluid, weighed and stored in phosphate buffer (pH=7.4) at −20° C. until assessed for drug concentration by HPLC.

Measurement of CsA levels in blood and tissue samples by HPLC—A published method was used for analysis of blood, plasma and tissue samples, with minor modifications¹⁰. Tissue samples were homogenized in phosphate buffer before analysis. To 200 μL of blood or tissue samples in a glass tube, 30 μL of internal standard (amiodarone, 10 μg/mL), 1.75 mL of deionised water, and 200 μL of sodium hydroxide (1 M) were added. The drug and internal standard were then extracted into 8 mL of an ether-methanol (95:5) solution by vortex mixing for 30 s. After centrifugation at 14,000 rpm for 5 min, the organic layer was removed and evaporated in vacuum (ISS110 Speedvac system, Thermosavant). The residue was then reconstituted in 200 μL of acetonitrile-0.5% (v/v) phosphoric acid (65:35). This solution was vortexed with 1 mL hexane for 10 s and centrifuged at 14,000 rpm for 2 min. Samples of 30 AL from the aqueous lower layer were injected into the HPLC system, (Waters 600 Multisolvent Delivery System and Waters 717 plus Autosampler) which was equipped with an LC₁ column (Supleco) with a mobile phase of KH₂PO₄ (0.01 M), methanol and acetonitrile (25:50:25). The flow rate and column temperature were set at 1 mL/min and 65° C. (Waters temperature control module), respectively. CsA concentrations were determined by UV detection at 205 nm (Waters 486). For quantization in tissues, concentration ranges of 1 to 40 mg/mL were employed in the calibration samples. For blood and plasma, the calibration samples were prepared at a concentration range of 0.1 to 10 mg/mL.

Pharmacokinetic Analysis—To account for the difference in dose between the two groups, blood, plasma and tissue concentrations for the test group (polymeric micellar formulation) were normalized by multiplying each concentration to the ratio of injected CsA dose for Cremophor formulation over injected CsA dose for polymeric micellar formulation. Pharmacokinetic of CsA has been shown to be linear at the administered CsA dose range of this study (≦5 mg/Kg) in Sprague Dawley rats¹¹. The elimination rate constant (λ_{z}) was estimated by linear regression of the blood concentrations in the log-linear terminal phase. In order to estimate the initial blood concentration (C₀) immediately after iv injection, the linear regression of the log-linear initial state going through the first two time points was extrapolated to the time zero. The estimated C₀ was then used with the actual measured plasma concentrations to determine the area under the blood concentration-time curve (AUC). The AUC_{inf} was calculated using the combined log-linear trapezoidal rule for data from time of dosing to the last measured concentration, plus the quotient of the last measured concentration divided by λ_{z}. Noncompartmental pharmacokinetic methods were used to calculate mean residence time (MRT by dividing AUMC_{inf} by AUC_{inf}) clearance (CL by dividing dose by AUC_{inf}), and volume of distribution (Vd by dividing CL by λ_{z}; and Vdss by multiplying CL by MRT). Tissue to plasma concentration ratios (Kₚ) were calculated by dividing CsA concentration in each tissue to CsA concentration in plasma for individual animals in the biodistribution studies.

Statistical analysis—Compiled data were presented as mean±SD. Where feasible, the data were analysed for statistical significance by unpaired Students t test. For this purpose the level of significance was set at α=0.05. In the tissue distribution studies, the AUC could not be determined in individual rats due to the destructive study design. Therefore, to permit an estimate of the variance associated with the composite mean AUC, the approach outlined by Bailer was utilized, which incorporates partial AUC and variability associated with each of the mean concentrations at each sampling point^{12,13,14}. Pairwise comparisons of the AUC were then undertaken at α=0.05. The critical value of Z (Z_{crit}) for the two-sided test after Bonferroni adjustment was 2.24¹⁵, and the observed value of Z (Z_{obs}) was calculated as previously described.

Results

### Example 1

### Synthesis, Characterization and Assembly of PEO-b-PCL Block Copolymers

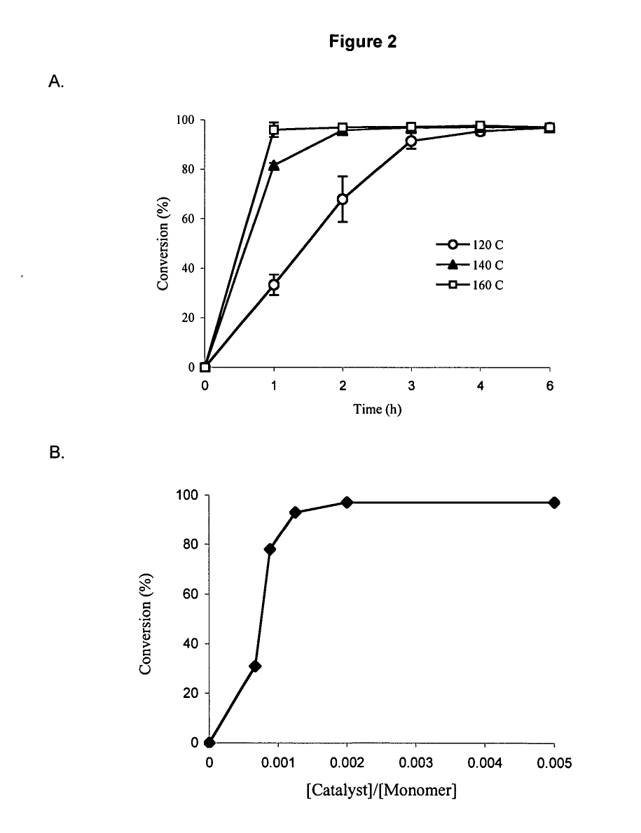

Synthesis of PEO-b-PCL block copolymers through ring opening polymerization of ε-caprolactone by methoxy PEO in the presence of stannous octoate has been reported before⁵. In the present study to determine optimal conditions, the catalyst level and temperature of the reaction were altered and the amount of residual monomer in the reaction product was measured over time by ¹H NMR. FIG. 2A illustrates the progress of polymerization for PEO-b-PCL block copolymers synthesized with a catalyst to monomer molar ratio of 0.002 at temperatures ranging between 120-160° C. When reaction temperatures were set at 120, 140 and 160° C., the maximum conversion of ε-caprolactone to PCL was achieved at 6, 3 and 2 hours, respectively. The effect of catalyst concentration on the monomer to polymer conversion was assessed in a second experiment when the reaction temperature and time were set at 140° C. and 4 hours, respectively. As shown in FIG. 2B, the highest conversion was achieved at catalyst to ε-caprolactone monomer molar ratios of more than 0.002. As a result, applying a temperature of 140° C. for 4 hrs and a catalyst to monomer ratio of 0.002 was chosen as an optimal condition for the preparation of PEO-b-PCL block copolymers. In the next step, the molar ratio of ε-caprolactone (monomer, M) to methoxy PEO (initiator, I) was changed to prepare PEO-b-PCL block copolymers having different degrees of ε-caprolactone polymerization. Prepared block copolymers were characterized for their average molecular weights by ¹H NMR and GPC. Considering a PEO molecular weight of 5000 g.mol⁻¹, at M/I molar ratios of 44, 114 and 210, PEO-b-PCL block copolymers with approximate PCL molecular weights of 5000, 13000 and 24000 g.mol⁻¹ were synthesized. A nomenclature of 5000-5000, 5000-13000 and 5000-24000 is used herein to distinguish between the PEO-b-PCL block copolymers with characteristics described in Table 1. As shown in Table 1, a good agreement between calculated and actual molecular weights was observed in most of the cases. Prepared block copolymers showed relatively narrow polydispersity indicating the absence of homopolymers (Table 1).

Micellization of PEO-b-PCL block copolymers was achieved through a co-solvent evaporation method. A solution of PEO-b-PCL block copolymers in acetone was added to water in a drop-wise manner followed by the evaporation of the organic solvent. The average diameter of PEO-b-PCL micelles prepared was between 78.7 and 99.8 nm, while Cremophor EL produced micelles with an average diameter of 11.3 nm (Table 2).

Low CMCs and high core viscosities were revealed for PEO-b-PCL micelles in fluorescent probe studies conducted using pyrene and 1,3-(1,1 dipyrenyl) propane (Table 2). Pyrene preferentially partitions into hydrophobic microdomains with a concurrent change in the molecule's photophysical properties. A sharp rise in intensity ratio of peaks at 337 nm to those at 333 nm from the excitation spectra of pyrene indicates the on-set of micellization (CMC) for block copolymers 16 Using this method, the average CMC for PEO-b-PCL block copolymers with 5000,13000 and 24000 g.mol⁻¹ of the PCL block was calculated at 1.8, 0.8 and 0.5 mg/mL, respectively (Table 2). A CMC value of 90 mg/mL for Cremophor EL has been reported in previous studies¹⁷. With pyrene as the fluorescent probe, the sharp rise in the intensity ratio of peaks at 337 nm to those at 333 nm from the excitation spectra was observed at a Cremophor EL concentration of 9.4 mg/mL. The fluorescent intensity ratio was levelled off at an approximate concentration of 100 mg/mL, which is close to reported CMC for Cremophor EL (data not shown). Changes in the excitation spectrum of pyrene in the presence of Cremophor EL at levels below its reported CMC may be attributed to the interaction of fluorescent probe with surfactant unimers or the existence of premicellar aggregates.

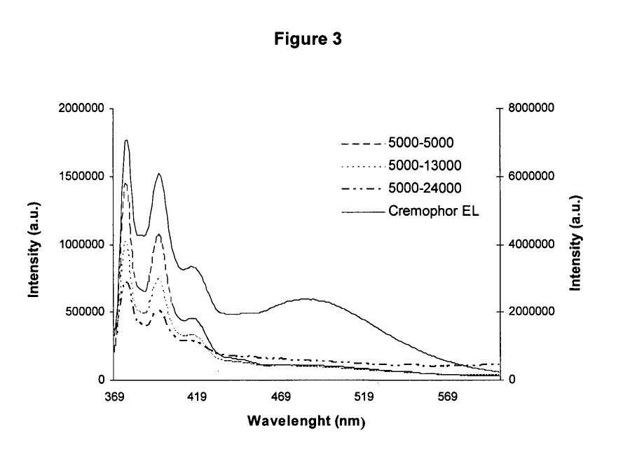

Very low Iₑ/Iₘ ratios (0.11-0.19) from the emission spectrum of 1,3-(1,1 dipyrenyl) propane for PEO-b-PCL micelles reflects a high viscosity for the PCL core. 1,3-(1,1 dipyrenyl) propane forms intramolecular pyrene excimers that emit light at 480 nm when excited at 390 nm. In a highly viscous environment, such as in the core of polymeric micelles, excimer formation is restricted. Emergence of a 1,3-(1,1 dipyrenyl) propane excimer peak at 480 nm for Cremophor EL reflects a lower micellar core viscosity for the low molecular weight surfactant (FIG. 3). Higher viscosity of the core in polymeric micelles may restrict the diffusion of the encapsulated drug leading to sustained drug release properties from the micellar carrier.

### Example 2

### Optimization of the Self-Assembly Process

Three different organic solvents were examined to find out the best solvent that can produce nanocarriers of less than 100 nm in diameter (Table 3). With THF, the size of the micelles was significantly larger and there were secondary peaks showing some degree of aggregation among the assembled micelles. The average diameter of micelles formed with acetonitrile and acetone were similar (82 and 89 nm) and showed narrow polydispersity. Evaporation of acetonitrile took longer than acetone, however.

The ratio of the two phases proved to be influential in the final characteristics of the micelles (Table 4). Using a lower ratio of the organic phase resulted in smaller micelles while the order of addition did not affect micellar size.

### Example 3

### Solubilization of CsA by PEO-b-PCL Micelles

Using an identical method to the self-assembly process, CsA was encapsulated into micelles of PEO-b-PCL. The level of encapsulated CsA was measured by HPLC after destroying the micellar structure with the aid of an organic solvent. CsA reached a level of 1.277 mg/mL (CsA: polymer weight ratio of 0.1277) in aqueous media by PEO-b-PCL micelles, and precipitated in water in the absence of the polymer (Table 5). Among PEO-b-PCL block copolymers of different PCL block lengths, maximum CsA: polymer weight ratio was achieved by PEO-b-PCL block copolymers with 13000 g.mol⁻¹ of the PCL block (Table 5). However, the molar CsA loading levels increased from 0.9 to 2.4 (mole CsA/mole polymer) with an increase in the molecular weight of the PCL block from 5000 to 24000 g.mol⁻¹. CsA encapsulation resulted in an increase in the average diameter of PEO-b-PCL micelles having 5000 and 13000 g.mol⁻¹ of PCL (Table 2 & 5). An increase in the initial level of applied drug from 2 to 3 mg did not change the solubilized CsA levels (Table 5).

### Example 4

### Optimization of CsA encapsulation

Acetone was chosen as the organic phase for drug loading by the co-solvent evaporation in PEO-b-PCL micelles. The effect of organic to aqueous phase ratio and order of phase addition on the level of CsA encapsulation at a block copolymer concentration of 10 mg/mL and CsA concentrations of 2 and 3 mg was evaluated.

The ratio of the two phases proved to be influential in the final CsA loading in PEO-b-PCL micelles, as well (Table 6). CsA reached a final concentration of 2 mg/mL in PEO-b-PCL micelles when the initial ratio of organic phase was reduced in the loading process. The order of phase addition on the other hand did not change drug loading and micellar characteristics.

### Example 5

### Characterization of CsA-Loaded PEO-b-PCL Micelles

In this study, water was replaced with normal saline to prepare isotonic polymeric micellar solutions of CsA for intravenous administration. When normal saline was used as the non-selective solvent, the efficiency of CsA encapsulation in PEO-b-PCL micelles was reduced (Table 7). In water, CsA reached an average aqueous concentration of 1.28 and 1.07 mg/mL applying initial CsA levels of 2 and 3 mg in the loading process, respectively. This level was reduced to an average concentration of 0.74 and 0.83 mg/mL when normal saline was used as the micellization medium. Premature precipitation of CsA during the micellization process in an acetone: normal saline solvent mixture in comparison to acetone: water environment might be the reason for lower levels of CsA encapsulation.

Replacement of water with normal saline did not affect the average diameter of empty and CsA-loaded PEO-b-PCL micelles (P>0.05, unpaired t test). Without CsA, the average micellar diameter was 78.7 and 79.8 nm in water and normal saline, respectively. The micellar size was raised to 118 nm in both solvents when 3 mg of CsA was added during the micellization process (Table 7).

### Example 6

### In vitro Release of CsA from Different Solubilizing Vehicles

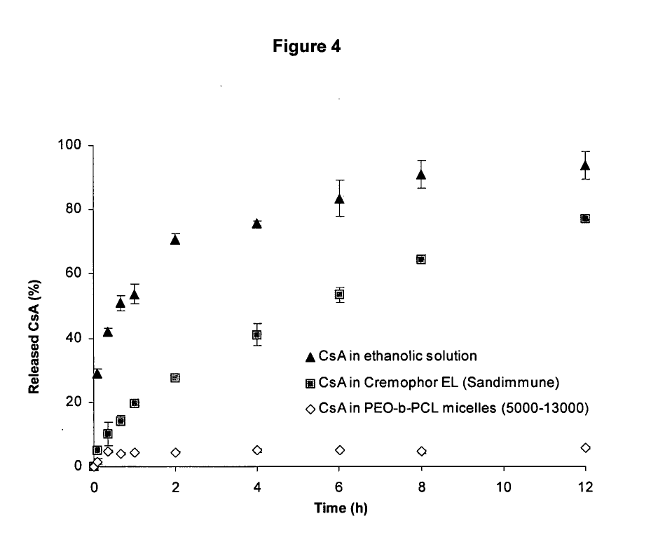

In a previous study, lipid vesicles have been used as the recipient phase in measuring the in vitro release rate of Amphotericin B from polymeric micelles¹⁸. In the case of CsA, lipid vesicles were found to be poor recipients, possibly due to the weak association of CsA with the lipid carrier (data not shown). Instead, BSA was used as a bio-mimetic recipient phase to maintain sink condition for the release of CsA from its vehicle. Encapsulated drug was separated from the recipient phase by a dialysis membrane having a molecular weight cut off of 12000-14000 g.mol⁻¹. Transfer of CsA through dialysis membrane to BSA was assumed to take place rapidly, and the release of CsA from its vehicle was assumed to be the rate limiting step in this process. In fact, 71% of un-encapsulated CsA from its ethanolic solution was transferred to BSA within 2 hours at 37° C. (FIG. 4). The release of CsA from Cremophor EL micelles (Sandimmune®) was found to be slower (27% of CsA was released in 2 hours). PEO-b-PCL micelles have sustained the rate of CsA release the most. After 12 hours, Cremophor EL formulation released 77% of its drug content while polymeric micellar vehicle released only 5.8% of encapsulated CsA. An increase in the molecular weight of PCL block from 5000 to 13000 g.mol-¹ had no significant effect on the rate of CsA release from PEO-b-PCL micelles within the time frame of this study.

### Example 7

### Pharmacokinetics of Polymeric Micellar CsA After Intravenous Administration

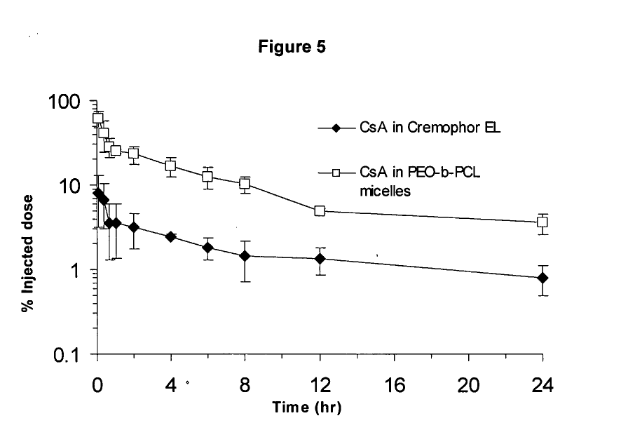

Linearity in the standard curves were demonstrated for each of the tissues over the concentration range studied, and chromatograms were free of interference from endogenous components. The blood CsA concentration vs. time profiles observed for both formulations were similar (FIG. 5). A rapid decline in concentrations, representing a distribution phase, was observed for the first 2 h after dosing. This was followed by an elimination phase with t_{1/2} of 9 to 12 h. However, after dose normalization, CsA in PEO-b-PCL micelles yielded higher blood concentrations than did the Cremophor formulation. Non-compartmental analysis of the blood concentrations showed a significant change in pharmacokinetic parameters of CsA in polymeric micelles in comparison to the Cremophor EL formulation (Table 8). PEO-b-PCL micelles provided significantly higher (6.1 fold) blood AUC compared to the Cremophor EL formulation. The PEO-b-PCL micelles also significantly decreased the volume of distribution (Vdss) and clearance (CL) of CsA by 10.0 and 7.6 fold, respectively. On the other hand, the terminal phase half life of CsA did not differ significantly between the two formulations.

### Example 8

### Tissue Distribution of Polymeric Micellar CsA in Healthy Animal Models

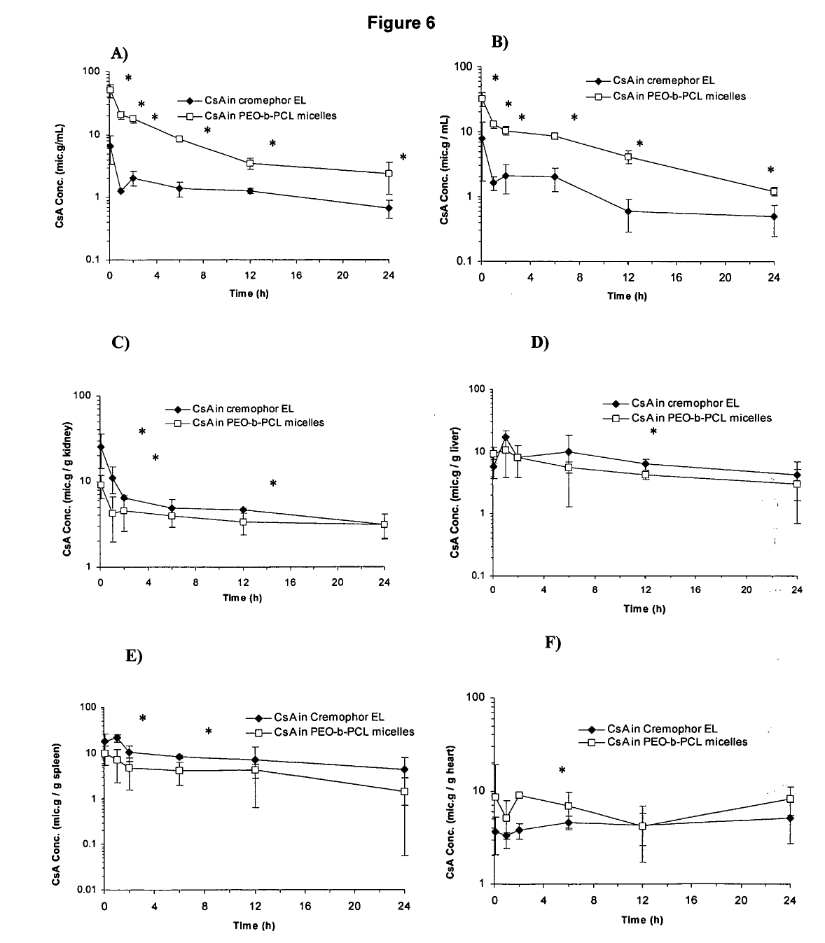

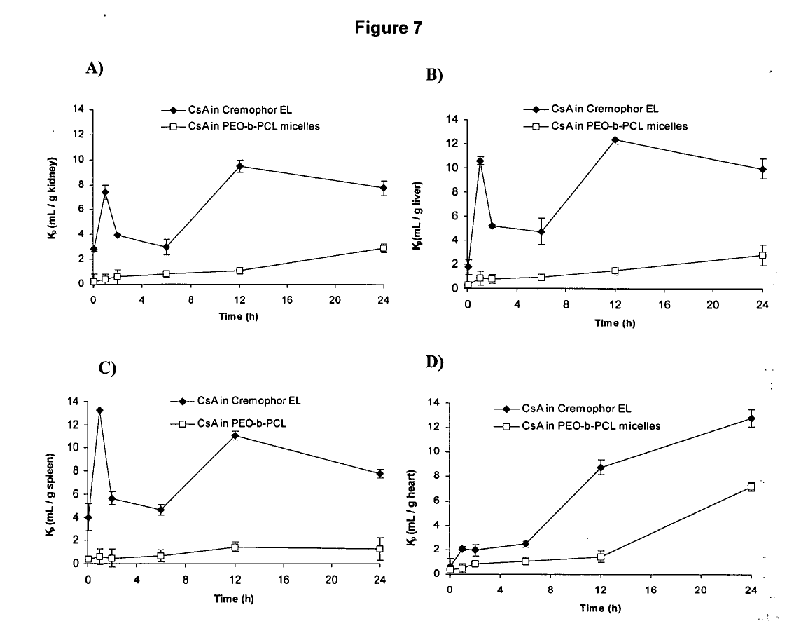

As in the pharmacokinetic study described above, a marked distribution phase was noted not only in the blood, but also in plasma (FIG. 6A & B). Upon intravenous administration of the Cremophor formulation, the mean dose normalized AUC in blood was comparable to that observed in the pharmacokinetic study. In the solid tissues assayed for drug content, quantifiable amounts of drug were not observed in the adipose or brain tissues. In those tissues where the AUC could be determined, the order in AUC from highest to lowest for the Cremophor formulation was spleen>liver>kidney>heart>blood>plasma (Table 9). In contrast, the corresponding order for the PEO-b-PCL micellar formulation was blood>heart>plasma>liver>kidney>spleen. Polymeric micelles showed 1.5, 1.4 and 2.1-fold lower AUCs of CsA in liver, kidney and spleen, respectively, when compared to Cremophor formulation. On the other hand, the AUC of CsA in the PEO-b-PCL micellar formulation was 5.7 and 4.9 times higher in blood and plasma, respectively.

Consistent with the pharmacokinetic experiments in rats in which serial blood collection was obtained (FIG. 5, Table 8), in the biodistribution study the blood and plasma concentrations and resultant AUC of CsA were significantly higher for the micellar formulation of the invention (FIG. 6, Table 9) than the available commercial formulation. There were some formulation-related differences detected at some discreet CsA concentrations in kidney, spleen, liver and heart (FIG. 6). Furthermore, in the tissues, a significantly lower AUC of CsA after administration of the micellar formulation was detected in spleen and kidney (Table 9). Heart, however, displayed a higher AUC after administration of the PEO-b-PCL micellar CsA formulation.

Variations in the tissue to plasma concentration ratios (Kₚ) of CsA were followed over time for both formulations (FIG. 7). The resultant profiles between the polymeric micellar formulation and Cremophor EL formulation were quite different. Compared to the commercial formulation, polymeric micelles showed much lower Kₚ values in kidney, liver, spleen and heart at all time points. Tissue to plasma ratios of polymeric micellar CsA remained below one for all organs up to 6 h and gradually increased afterwards (FIG. 7A-C), except for the heart in which a sharp raise after 12 h has been observed (FIG. 7D). In kidney, liver and spleen, the Kₚ ratio for the commercial formulation of CsA demonstrated a biphasic pattern with maximum values at one and 12 h after injection. One h after intravenous injection of Sandimmune®, the Kₚ ratio was 7.4, 10.6, 13.3 and 2.1 for kidney, liver, spleen and heart, respectively, compared to Kₚ values of 0.4, 0.9, 0.6 and 0.5 for the polymeric micellar formulation.

Discussion

The present invention relates to a novel delivery system for hydrophobic drug solublization, said system allowing control of the rate of drug release and disposition in a biological system and is safe for human administration. Micelles of methoxy poly(ethylene oxide)-b-poly(ε-caprolactone) (PEO-b-PCL) were chosen as potential carrier for this purpose due to the biocompatibility and biodegradability of the PEO and PCL blocks, thermodynamic stability of the micellar structure, and distinct properties of the PEO/PCL segments.

Synthesis of PEO-b-PCL was carried out through ring opening polymerization of ε-caprolactone in the presence of stannous octoate as catalyst. Yuan et al have reported on the application of a similar process at a temperature of 140° C. for 24 h¹⁴. In the present study, reaction conditions for the preparation of PEO-b-PCL block copolymers through ring opening polymerization were optimized with respect to time, temperature and catalyst concentration. Inadequate time or temperature of the reaction in the ring opening polymerization of lactones may lead to incomplete conversion of the monomer to polymer¹⁹, whereas long reaction times or high temperatures may result in transesterification or back biting degradation of the polyester chain leading to an increase in the polydispersity of the prepared block copolymers¹⁴. At a reaction temperature of 140° C. and a reaction period of 4 hrs (instead of 24 h) sufficient conversion of ε-caprolactone to PCL occurred (FIG. 2A). Block copolymers synthesized at this condition showed better polydispersity compared to what has been reported earlier¹⁴ (Table 1). A reaction temperature of 160° C. for 3 h was also shown to be sufficient for optimum conversion of ε-caprolactone to PCL (FIG. 2).

Prepared block copolymers were assembled to micellar structures by a co-solvent evaporation method and characterized for their functional properties in drug delivery. Self assembly of block copolymers may be accomplished through direct dissolution²⁰, solvent evaporation/film formation²¹ or dialysis²² methods. Direct dissolution and solvent evaporation/film formation were shown to be less optimal methods for the self assembly of PEO-b-PCL block copolymers, especially for those with long PCL chain lengths, because of the high hydrophobicity of the PCL. Preparation of PEO-b-PCL micelles through the evaporation of a co-solvent azeotrope (acetonitrile/water) has been reported by Kwon et al²³. In the current study, an acetone/water co-solvent system has been used to match the higher hydrophobicity of the core-forming block used here. Recently, tetrahydrofuran (THF)/water co-solvent systems have been used by Gao et al. for the micellization of PEO-b-PCL block copolymers having long PCL blocks (PCL molecular weights of 2500 to 24700 g.mol⁻¹)²⁴. The acetone: water system may be beneficial in terms of scale-up because of a lower boiling point, however.

A comparison between various studies on the preparation of PEO-b-PCL micelles demonstrates that aside from block copolymer molecular weight other factors such as micellization procedure or solvent composition play a role in determining the average diameter and size distribution of assembled nano-carriers^{21,23,24,25,26}. Using acetone: water (1:2) system, PEO-b-PCL block copolymers with a PEO molecular weight of 5000 g.mol⁻¹ and PCL molecular weights of 5000 to 24000 g.mol⁻¹ produced polymeric micelles with an average diameter of 79-100 nm (Table 2). At a similar block copolymer molecular weight range, application of a THF: water (1:10) system has resulted in the formation of smaller micelles (an average diameter of 41-86 nm)²⁴. The difference in size is specially marked for self-assembled structures formed from 5000-5000 PEO-b-PCL block copolymers (an average diameter of 41.0 nm for THF: water versus a diameter of 87.5 nm for acetone: water system). Although the size distribution of the self-assembled structures prepared from 5000-5000 PEO-b-PCL micelles in the acetone: water co-solvent mixture in this study were unimodal, a higher polydispersity index (Table 2) might be an indication of a trend towards aggregate formation with a larger average diameter of this particular structure. Application of dialysis methods for the self-assembly of PEO-b-PCL block copolymers having similar PCL chain lengths has resulted in the formation of larger particles, even when THF has been used as the selective solvent for the core-forming block in the micellization of PEO-b-PCL²⁵.

Micellar characteristics and drug loading levels may be optimized through changes in the self assembly process. Examples 2 and 4, and corresponding Tables 3, 4 and 6, show that, for CsA loading in PEI-b-PCL micelles, optimum drug solublization and micellar size can be achieved with the addition of acetone to water at a final organic to aqueous phase ration of 1:6.

The acetone: water co-solvent evaporation procedure was efficient for the encapsulation of CsA in PEO-b-PCL micelles (Table 6). Aqueous solubility of CsA was increased up to 50 fold, reaching to a level of 1 mg/mL, in the presence of PEO-b-PCL micelles. This level is much higher than the water solubility of CsA (23 μg/mL) and is considered relevant for clinical application. This level is also comparable to injected CsA concentrations in Sandimmune®, which is between 0.5-2.5 mg/mL. To provide a CsA aqueous concentration of 0.5-2.5 mg/mL, 6.5-32.5 mg/mL of Cremophor EL is required in the Sandimmune® formulation. This level corresponds to a drug to vehicle loading weight ratio of 0.078 mg/mg. To achieve a similar CsA level, the polymeric micellar formulation requires 10 mg/mL of PEO-b-PCL, which corresponds to drug to vehicle loading weight ratios of 0.09-0.13 mg/mg for block copolymers of various PCL lengths.

Compared to Cremophor EL, even lower CMC (Table 2) and higher core viscosity, characteristic of the polymeric structure of the hydrophobic domain, were revealed for PEO-b-PCL micelles (FIG. 3). The lower CMCs reflect the thermodynamic stability of micellar structure, allowing a lower dose of injection to keep block copolymers above the CMC and in a micellar form upon dilution in blood after intravenous administration. The low value of the excimer to monomer intensity ratios (Iₑ/Iₘ) indicated that PEO-b-PCL micellar cores have a high viscosity that may postpone the dissociation of micellar structure into polymeric unimers below the CMC.

Further effort for the optimization of the polymeric micellar formulation of CsA was carried out through changes in the length of the PCL block. A reverse relationship between the hydrophobicity of the core-forming block and CMC, and a direct relationship between this factor and CsA loading (on molar basis) has been observed. A decrease in the CsA loading weight ratio (weight of loaded CsA/weight of polymer) in 5000-24000 PEO-b-PCL micelles may reflect the lower number of micelles present for this block copolymer at a copolymer concentration of 10 mg/mL.

Finally, unlike the Cremophor EL micelles that have mobile cores, controlled CsA release has been achieved with PEO-b-PCL micelles. PEO-b-PCL 94% of their drug content after 12 hrs (FIG. 4).

In accordance with the high viscosity of the core in polymeric micelles, the in vitro rate of CsA transfer to bovine serum albumin was remarkably sustained by PEO-b-PCL micelles. Micelles of Cremophor EL, on the other hand, showed liquid like structures and released 95% of their drug content within 24 h²⁷.

In terms of blood, plasma and tissue CsA concentrations, the novel PEO-b-PCL micellar formulation of CsA demonstrated some significant differences from the commercially available Cremophor formulation. The 6.1-fold higher AUC in whole blood is reflective of a high degree of in vivo stability of the polymeric micellar particles. This is a significant finding, because lipid based carriers have typically failed to show meaningful changes in the pharmacokinetics of encapsulated CsA^{28,29,30}. In lipid based delivery systems, weak CsA binding to the lipid membrane appears to cause a premature leakage of the encapsulated drug from the carrier^{29,30,31}.

The change in the biological fate of CsA, imposed by its encapsulation in PEO-b-PCL micelles, has led to an increase in AUC as a result of a reduction in CL and Vd for the encapsulated drug. This is in contrast to the results of pharmacokinetic studies by Kim et al³², who recently reported a decrease in AUC and an increase in the Vd and half life, but no change in the CL of indomethacin loaded PEO-b-PCL micelles in Sprague-Dawley rats. While not wishing to be limited by theory, stealth properties of the developed micellar carrier in the present study, induced by the nanoscopic dimension and hydrophilic shell of PEO might have contributed to the present observation.

In this study, with the exception of heart, the increase in blood CsA concentrations seemed to coincide with a decrease in the tissue concentrations of the drug, which is in line with the reduced Vd after administration of the PEO-b-PCL formulation. Using Bailer's approach to statistical analysis of the AUC, significance was established for a decrease of splenic and renal uptake of CsA, and there were some discreet concentrations in liver that showed a significant decrease in CsA uptake for the micellar formulation. In general, CsA encapsulation in PEO-b-PCL micelles resulted in elevated CsA levels in blood, plasma and heart, but appeared to reduce CsA uptake by kidney, liver and spleen. This shift in concentrations from kidney to blood is of note, because one of the major dose-limiting side effects of CsA is kidney toxicity. The trend towards accumulation of polymeric micellar CsA in heart is an interesting observation that, while not wishing to be limited by theory, might have been caused by preferential accumulation of the polymeric micellar carrier in heart or avoidance of efflux mechanisms by encapsulated CsA in this organ.

The Kₚ value for CsA were lowered in kidney, spleen and liver by PEO-b-PCL micelles especially at early time points, indicating the localization of the micellar carrier in the plasma compartment after iv administration. In contrast, the Cremophor EL formulation showed high distribution of CsA in all organs. The maximum value of Kₚ observed in kidney, liver and spleen one hour after iv administration of this formulation, reflects the rapid distribution of CsA in those organs. Again, while not wishing to be limited by theory, an increase in the Kₚ value of CsA-loaded PEO-b-PCL micelles in kidney, liver and heart after 12 h, which is a result of reduced CsA concentrations in plasma, may reflect the destabilization of the micellar formulation in plasma.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Characteristics of the prepared PEO-b-PCL block copolymers.

| | PCL | | PEO-b- | PEO-b- | PEO-b-PCL |
|---|---|---|---|---|---|
| PEO Mwt. | M. Wt | | PCL | PCL | Polydispersity |
| (g · mol⁻¹) | (g · mol⁻¹)^{a} | [M]/[I]^{b} | Mₙ^{c} | Mₙ^{d} | (M_{w}/Mₙ)^{d} |
| 5000 | 5000 | 44 | 9984 | 11007 | 1.035 |
| 5000 | 13000 | 114 | 17318 | 23008 | 1.063 |
| 5000 | 24000 | 210 | 27800 | 30035 | 1.065 |

^{a}Theoretical molecular weight;
^{b}Monomer to initiator molar ratios
^{c}Determined by ¹H NMR;
^{d}Determined by GPC.

TABLE 2
Characteristics of Cremophor EL and PEO-b-PCL
micelles with different core structures.

| Amphiphilic | Micellar size ± SD | Polydispersity | CMC ± SD |
|---|---|---|---|
| monomer | (nm) | Index | (mg/mL) |
| Cremophor EL | 11.3 ± 0.2 | 0.108 | 90^{a} |
| PEO-b-PCL | 87.5 ± 9.6 | 0.198 | 1.8 ± 0.1^{b} |
| (5000-5000) | | | |
| PEO-b-PCL | 78.7 ± 11.8 | 0.111 | 0.8 ± 0.1^{b} |
| (5000-13000) | | | |
| PEO-b-PCL | 99.8 ± 3.8 | 0.119 | 0.5 ± 0.2^{b} |
| (5000-24000) | | | |

^{a}Reported from reference [28]
^{b}Measured from the onset of a rise in the intensity ratio of peaks at 337 nm to peaks at 333 nm in the fluorescence spectra of pyrene plotted versus logarithm of polymer concentration.

TABLE 3
Micellar size and polydispersity Index of prepared
micelles in different organic phases (10 mg/mL
of the polymers with M. wt of 18,000)

| Organic Phase | Size ± SD | PI |
|---|---|---|
| Acetone* | 87.8 ± 9.4 | 0.111 |
| Acetonitrile* | 82.9 ± 12.27 | 0.104 |
| THF* | 295.5^{†} | 0.523 |

*A ratio of 1:2 of organic:aqueous phases were used, and the organic phase was added to the aqueous phase.
^{†}A secondary peak was observed in 2 out of three experiments at 446.4 and 1712.2 nm.

TABLE 4
Micellar size and polydispersity Index of co-polymeric
micelles prepared by two methods: addition of organic
phase to aqueous phase and vise versa, and the effect
of organic:aqueous phase ratio on the micellar size

| Acetone: | Acetone to aqueous phase | | Water to acetone solution | |
|---|---|---|---|---|
| Water ratio | Size ± SD | PI | Size ± SD | PI |
| 1:2 | 87.8 ± 9.4 | 0.111 | 74.87 ± 1.92 | 0.0875 |
| 1:6 | 63 ± 4.00 | 0.142 | 57.2 ± 9.79 | 0.117 |

TABLE 5
Effect of the order of addition, and ratio of phases on loading
efficiency of CsA in PEO-b-PCL micelles (5,000-13,000)

| | Ratio | CsA initial | CsA | Encapsulation | | |
|---|---|---|---|---|---|---|
| Order of | (Organic: | Conc. | loading ± | Efficiency ± β | Micellar | |
| Addition | Aqueous) | (mg/mL) | SD (w/w) | SD (%) | size (nm) | PI |
| Acetone | 1:2 | 3 | 0.1071 ± 0.0072 | 35.7 ± 2.4 | 108.9 ± 25.00 | 0.121 |
| to water | 1:6 | 3 | 0.1970 ± 0.0274 | 65.65 ± 9.13 | 89.33 ± 15.30 | 0.207 |
| Water to | 1:2 | 3 | 0.1483 ± 0.0132 | 49.43 ± 4.40 | 94.43 ± 10.96 | 0.180 |
| acetone | 1:6 | 3 | 0.1496 ± 0.0147 | 49.87 ± 4.90 | 66.88 ± 6.01 | 0.210 |

TABLE 6
Encapsulation of CSA in PEO-b-PCL micelles:
Effect of PCL chain length.

| | | | CsA: | |
|---|---|---|---|---|
| PEO-PCL | Encapsulated | | Polymer | Micellar |
| Mwt. | CsA | Encapsulation | Ratio | size (nm) |
| (g · mol⁻¹) | (mg/mL) | Efficiency (%) | (M:M) | (Z Ave) |
| 10,000 | 1.0 | 52 | 1.7 | 100 |
| 18,000 | 1.3 | 67 | 2.0 | 121.2 |
| 24,000 | 1.1 | 56 | 1.7 | 84 |

TABLE 7
Characteristics of CsA-loaded PEO-b-PCL micelles with respective PEO
and PCL molecular weights of 5000 and 13000 g · mol⁻¹.

| | Initial | Encapsulated | | CsA: | Size of | Size of |
|---|---|---|---|---|---|---|
| Medium | CsA | CsA level ± | Encapsulation | Polymer | drug free | CsA-loaded |
| of | level | SD | Efficiency ± | ratio ± | micelles ± | micelles ± |
| Micellization | (mg) | (mg/mL) | SD (%) | SD (M:M) | SD (nm) | SD (nm) |
| Water^{a} | 2 | 1.277 ± 0.131 | 63.8 ± 6.57 | 1.91 ± 0.20 | 78.7 ± 11.8 | 98.6 ± 13.2 |
| Normal saline | 2 | 0.740 ± 0.022 | 37.0 ± 1.11 | 1.11 ± 0.03 | 79.8 ± 7.79 | 105 ± 4.42 |
| Water^{a} | 3 | 1.071 ± 0.072 | 35.7 ± 2.41 | 1.60 ± 0.11 | 78.7 ± 11.8 | 118 ± 16.7 |
| Normal saline | 3 | 0.825 ± 0.143 | 27.5 ± 4.79 | 1.23 ± 0.21 | 79.8 ± 7.79 | 118 ± 16.2 |

^{a}reproduced from ref [27]

TABLE 8
Noncompartmental pharmacokinetic parameters (±SD) of CsA after
intravenous administration of CsA in polymeric micellar formulation
in comparison to CsA in CremophorEL (Sandimmune ®) formulation.
Micellar data is normalized to injected dose of Cremophor EL and
presented as mean ± SD (n = 4).

| | CsA in | CsA in |
|---|---|---|
| | CremophorEL | polymeric micelles |
| AUC₀₋₂₄ (mg · h/mL) | 25.3 ± 7.64 | 167 ± 18.8 ^{a} |
| AUC_{0-∞}(mg · h/mL) | 32.7 ± 13.8 | 199 ± 20.9 ^{a} |
| t_{1/2} (hr) | 11.5 ± 4.58 | 9.40 ± 1.20 |
| MRT (hr) | 14.4 ± 6.62 | 9.24 ± 2.06 |
| CL (L/kg/h) | 0.195 ± 0.131 | 0.0255 ± 0.00319 ^{a} |
| Vdₛₛ (L/kg) | 2.33 ± 0.785 | 0.232 ± 0.0425 ^{a} |

^{a} Denotes significant difference between groups.

TABLE 9
Area under the blood, plasma and tissue concentration time
curves of CsA after IV administration of CsA- loaded polymeric
micelles and CsA in CremophorEL (Sandimmune ®) formulation.
Micellar data is normalized to injected dose of Cremophor
EL and presented as mean ± SD.

| | CsA in CremophorEL | CsA in polymeric micellar |
|---|---|---|
| Specimen | formulation (mg · h/g) | formulation (mg · h/g) |
| Blood | 31.8 ± 1.59 | 181 ± 8.07^{a} |
| Plasma | 29.5 ± 3.23 | 143 ± 5.61^{a} |
| Kidney | 128 ± 7.12 | 91.4 ± 6.12^{a} |
| Liver | 176 ± 27.5 | 119 ± 8.52 |
| Spleen | 188 ± 32.1 | 90.0 ± 10.1^{a} |
| Heart | 107 ± 13.8 | 155 ± 13.4^{a} |

^{a}Denotes significant difference between groups.

### FULL CITATIONS FOR REFERENCES REFERRED TO IN THE SPECIFICATION

¹Zastre J. Jackson J. Bajwa M. Liggins R. Iqbal F. Burt H European Journal of Pharmaceutics & Biopharmaceutics. Vol 54(3) (pp 299-309), 2002.
²Kim S Y. Lee Y M. Taxol-loaded block copolymer nanospheres composed of methoxy poly(ethylene glycol) and poly(epsilon-caprolactone) as novel anticancer drug carriers. Biomaterials. Vol22(13) (pp 1697-1704),2001.
³Shin, I. G., et al., Methoxy poly(ethylene glycol)/epsilon-caprolactone amphiphilic block copolymeric micelle containing indomethacin. I. Preparation and characterization. Journal of Controlled Release, 1998. 51(1): p. 1-11.
⁴Allen, C., et al., Polycaprolactone-b-poly(ethylene oxide) block copolymer micelles as a novel drug delivery vehicle for neurotrophic agents FK506 and L-685,818. Bioconjugate Chemistry, 1998. 9(5): p. 564-72.
⁵Yuan, M., et al., Polymerization of lactides and lactones.10. Synthesis, characteization, and application of amino terminated poly(ethylene glycol)-co-poly(ε-caprolactone) block copolymer. Macromolecules, 2000. 33: p. 1613-1617.
⁶Stridsberg K. M. et al. “Controlled ring-opening polymerization: Polymers with designed macromolecular architecture” Advanced Polymer Science, (2002) 157: 42-62.
⁷H. Montazeri Aliabadi, D. R. Brocks, A, Lavasanifar, Polymeric micelles for the delivery of cyclosporine A: Pharmacokinetics and biodistribution, Biomaterials 2005, in press.
⁸A. Lavasanifar, J. Samuel, S. Sattari and G. S. Kwon Block copolymer micelles for the encapsulation and delivery of amphotericin b, Pharmaceutical Research 19 (2002) 418-422.
⁹Brocks, D R, Betageri, G V, Enhanced oral absorption of halofantrine enantiomers after encapsulation in a proliposomal formulation. J Pharm Pharmacol 2002; 54(8): 1049-53.
¹⁰Chimalakonda, A P, Shah, R B, Mehvar, R, High-performance liquid chromatographic analysis of cyclosporin A in rat blood and liver using a commercially available internal standard. J Chromatogr B Analyt Technol Biomed Life Sci 2002; 772(1): 107-14.
¹¹Tanaka, C, Kawai, R, Rowland, M, Dose-dependent pharmacokinetics of cyclosporin A in rats: Events in tissues. Drug Metab Dispos 2000; 28(5): 582-9.
¹²Mehvar, R, Robinson, M A, Reynolds, J M, Molecular weight dependent tissue accumulation of dextrans: In vivo studies in rats. J Pharm Sci 1994; 83(10): 1495-1499.
¹³Bailer, A J, Testing for the equality of area under the curves when using destructive measurement techniques. J Pharmacokinet Biopharm 1988; 16(3): 303-309.
¹⁴Yuan, J, Estimation of variance for AUC in animal studies. J Pharm Sci 1993; 82(7): 761-763.
¹⁵Miller, R G, In: Simultaneous statistical inference. McGraw-Hill series in probability and statistic. 1966, USA: McGraw-Hill. 272.
¹⁶M. Wilhelm, C. L. Zhao, Y. Wang, R. Xu, M. A. Winnik, J. L. Mura, G. Riess and M. D. Croucher Poly(styrene-ethylen oxide) block copolymer micelle formation in water: A flourescence probe study, Macromolecules 24 (1991) 1033-1040.
¹⁷D. Kessel Properties of cremophor el micelles probed by fluorescence, Photochemistry & Photobiology 56 (1992) 447-451.
¹⁸A. Lavasanifar, J. Samuel and G. S. Kwon The effect of fatty acid substitution on the in vitro release of amphotericin b from micelles composed of poly(ethylene oxide)-block-poly(n-hexyl stearate-I-aspartamide), J Control Release 79 (2002) 165-172.
¹⁹R. T. Liggins and H. M. Burt Polyether-polyester diblock copolymers for the preparation of paclitaxel loaded polymeric micelle formulations, Adv Drug Deliv Rev 54 (2002) 191-202.
²⁰C Allen, D. Maysinger and A. Eisenberg Nano-engineering block copolymer aggregates for drug delivery, Colloids & Surfaces B: Biointerfaces 16 (1999) 3-27.
²¹A. Lavasanifar, J. Samuel and G. S. Kwon Micelles self-assembled from poly(ethylene oxide)-block-poly(n-hexyl stearate I-aspartamide) by a solvent evaporation method: Effect on the solubilization and haemolytic activity of amphotericin b, J Control Release 77 (2001) 155-160.
²²C Allen, J. Han, Y. Yu, D. Maysinger and A. Eisenberg Polycaprolactone-b-poly(ethylene oxide) copolymer micelles as a delivery vehicle for dihydrotestosterone, Journal of Controlled Release 63 (2000) 275-286.
²³K. K. Jette, D. Law, E. A. Schmitt and G. S. Kwon Preparation and drug loading of poly(ethylene glycol)-block-poly(epsilon-caprolactone) micelles through the evaporation of a cosolvent azeotrope, Pharm Res 21 (2004) 1184-1191.
²⁴X. Shuai, H. Ai, N. Nasongkla, S. Kim and J. Gao Micellar carriers based on block copolymers of poly(epsilon-caprolactone) and poly(ethylene glycol) for doxorubicin delivery, Journal of Controlled Release 98 (2004) 415-426.
²⁵S. Y. Kim, I. G. Shin, Y. M. Lee, C. S. Cho and Y. K. Sung Methoxy poly(ethylene glycol) and epsilon-caprolactone amphiphilic block copolymeric micelle containing indomethacin. Ii. Micelle formation and drug release behaviours, Journal of Controlled Release 51 (1998) 13-22.
²⁶X Shuai, T. Merdan, A. K. Schaper, F. Xi and T. Kissel Core-cross-linked polymeric micelles as paclitaxel carriers, Bioconjugate Chemistry 15 (2004) 441-448.
²⁷Montazeri Aliabadi, H, Mahmud, A, Dehmoobed Sharifabadi, A, Lavasanifar, A, Micelles of methoxy poly(ethylene oxide)-b-poly(ε-caprolactone) as vehicles for the solublization and controlled delivery of cyclosporin A. J. Control. Release 2005; 104(2) 301-311.
²⁸Lee, M K, Choi, L, Kim, M H, Kim, C K, Pharmacokinetics and organ distribution of cyclosporin A incorporated in liposomes and mixed micelles. Int J Pharm. 1999; 191(2): 87-93.
²⁹Fahr, A, Holz, M, Fricker, G, Liposomal formulations of cyclosporin A: Influence of lipid type and dose on pharmacokinetics. Pharm Res 1995; 12(8): 1189-98.
³⁰Choice, E, Liposomal cyclosporine. Comparison of drug and lipid carrier pharmacokinetics and biodistribution. Transplantation 1995; 60(9): 1006-11.
³¹Ouyang, C, Choice, E, Holland, J, Meloche, M, Madden, T D, Liposomal cyclosporine. Characterization of drug incorporation and interbilayer exchange. Transplantation 1995; 60(9): 999-1006.
³²Kim, S Y, Lee, Y M, Shin, H J, Kang, J S, Indomethacin-loaded methoxy poly(ethylene glycol)/poly(epsilon-caprolactone) diblock copolymeric nanosphere: Pharmacokinetic characteristics of indomethacin in the normal sprague-dawley rats. Biomaterials 2001; 22(14): 2049-56.

1. A PEO-b-PCL micelle comprising PEO-b-PCL block copolymer exhibiting a molecular weight of greater than 6000 Daltons.

2. The micelle according to claim 1, wherein the molecular weight of the copolymer is between 10,000-29,000 Daltons.

3. The micelle according to claim 2, wherein the molecular weight of the copolymer is about 18,000 Daltons.

4. The micelle according to claim 1, wherein the PEO molecular weight is 5000 Daltons or greater.

5. The micelle according to claim 1, wherein the PCL molecular weight is 5000 Daltons or greater.

6. The micelle according to claim 1, further comprising a biologically active agent.

7. The micelle according to claim 6, wherein the biologically active agent is hydrophobic.

8. The micelle according to claim 6, wherein the agent is selected from an organic or inorganic small molecule compound, a polymeric species, a lipid and mixtures thereof, wherein said agent is administered to subjects in vivo for the treatment of a disease, disorder or condition.

9. The micelle according to claim 8, wherein the polymeric species is selected from nucleic acids, proteins, peptides, carbohydrates and derivatives thereof.

10. The micelle according to claim 7, wherein the biologically active agent is selected from cyclosporin A, PSC 833, amphotercin B, nystatine, diazepam, amiodarone, verapamil, indomethacin, taxol, rapamycin, etoposide and estradiol.

11. The micelle according to claim 7, wherein the biologically active agent is cyclosporine A and/or one of its analogs.

12. The micelle according to claim 11, wherein the biologically active agent is cyclosporine A or PSC 833.

13. The micelle according to claim 6 wherein the biologically active agent is an inhibitor of P-glycoprotein.

14. The micelle according to claim 6 wherein the biologically active agent promotes immunosuppression or moderation of drug resistance in mammals.

15. The micelle according to claim 6, wherein the biologically active agent is used to treat resistant forms of cancer or infectious disease in mammals.

16. The micelle according to claim 6, wherein the biologically active agent is amiodarone.

17. (canceled)

18. A method for preparing PEO-b-PCL micelles comprising assembling a PEO-b-PCL block co-polymer in a suitable aqueous medium under conditions sufficient to minimize aggregation.

19. The method according to claim 18 comprising:
obtaining a solution of PEO-b-PCL block copolymers in a water miscible solvent;
combining the solution of PEO-b-PCL block copolymers with a suitable aqueous medium under conditions sufficient to minimize aggregation; and
removing the water miscible organic solvent.

20. The method according to claim 18 wherein the PEO-b-PCL block copolymer comprises PEO of molecular weight of greater than 5000 Daltons.

21. The method according to claim 18, wherein the PEO-b-PCL block copolymer comprises PCL of molecular weight of greater than 5000 Daltons.

22. The method according to claim 19, wherein the water-miscible solvent is acetone or acetonitrile.

23. The method according to claim 22 wherein the water-miscible solvent is acetone.

24. The method according to claim 19, wherein the aqueous medium is water, saline 5% dextrose or isotonic sucrose.

25. The method according to claim 19, wherein the ratio of the solution of PEO-b-PCL block copolymers to aqueous medium is between about 1:2 and about 1:10.

26. The method according to claim 25, wherein the ratio of the solution of PEO-b-PCL block copolymers to aqueous medium is about 1:6.

27. The method according to claim 19, wherein the solution of PEO-b-PCL block copolymers is added in dropwise fashion to the aqueous medium.

28. The method according to claim 18, wherein the micelle further comprises a biologically active agent and agent drug is added in step (a).

29. The method according to claim 18, wherein said drug is a cyclosporine.

30. The method according to claim 29, wherein said cyclosporine is cyclosporine A.

31. (canceled)

32. (canceled)

33. (canceled)

34. (canceled)

35. A method of delivering biologically active agents to treat a disease, condition or disorder in a subject in need thereof comprising administering effect amount of a biological agent-loaded micelle as claimed in claim 6 to said subject.

36. The method according to claim 35, wherein the agent is cyclosporine, CsA or analog thereof.

37. The method according to claim 36, wherein the disease, condition or disorder is one that benefits from the administration of CsA or an analog thereof.

38. The method according to claim 37, wherein said diseases, conditions or disorders is one that benefits from administration of an immunosuppressant.

39. (canceled)

40. The method according to claim 36, wherein the disease, condition or disorder is cancer or drug resistant cancers, infectious disease or an autoimmune disease.

41. (canceled)

42. (canceled)

43. (canceled)

44. (canceled)

45. (canceled)

46. (canceled)

47. (canceled)

48. (canceled)

49. The micelle according to claim 2, wherein the molecular weight of the copolymer is about 24,000 to about 29,000 Daltons.
